SEQUENCE LISTING (12 sequences)

SEQ ID NO: 1 - protein, 5 aa, synthetic construct
  REGION 1..5: note = 27A6 HCDR1
RYWIE

SEQ ID NO: 2 - protein, 17 aa, synthetic construct
  REGION 1..17: note = 27A6 HCDR2
EILPGSGTTNYNENFRG

SEQ ID NO: 3 - protein, 9 aa, synthetic construct
  REGION 1..9: note = 27A6 HCDR3
SYYGDFDAY

SEQ ID NO: 4 - protein, 14 aa, synthetic construct
  REGION 1..14: note = 27A6 LCDR1
RSSIGAVTTSNHAN

SEQ ID NO: 5 - protein, 7 aa, synthetic construct
  REGION 1..7: note = 27A6 LCDR2
GTNNRAP

SEQ ID NO: 6 - protein, 9 aa, synthetic construct
  REGION 1..9: note = 27A6 LCDR3
ALWYSNHWV

SEQ ID NO: 7 - protein, 118 aa, synthetic construct
  REGION 1..118: note = 27A6 VH
QVQLQQSGAELMEPGASVKISCKATGYTFSRYWIEWVQQRPGHGLEWIGEILPGSGTTNYNENFRGKATFTADTSSNTVYMQLISLTSEDSAVYYCARSYYGDFDAYWGQGTLVTVSA

SEQ ID NO: 8 - protein, 109 aa, synthetic construct
  REGION 1..109: note = 27A6 VL
QAVVTQESALTTSPGETVTLTCRSSIGAVTTSNHANWVQEKPDHLFTGLIGGTNNRAPGVPARFSGSLIGDKAVLTITGAQTEDEAIYFCALWYSNHWVFGGGTKLTVL

SEQ ID NO: 9 - protein, 324 aa, synthetic construct
  REGION 1..324: note = 27A6 heavy chain constant region
AKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 10 - protein, 106 aa, synthetic construct
  REGION 1..106: note = 27A6 light chain constant region
GQPKSSPSVTLFPPSSEELETNKATLVCTITDFYPGVVTVDWKVDGTPVTQGMETTQPSKQSNNKYMASSYLTLTARAWERHSSYSCQVTHEGHTVEKSLSRADCS

SEQ ID NO: 11 - protein, 442 aa, synthetic construct
  REGION 1..442: note = 27A6 heavy chain
QVQLQQSGAELMEPGASVKISCKATGYTFSRYWIEWVQQRPGHGLEWIGEILPGSGTTNYNENFRGKATFTADTSSNTVYMQLISLTSEDSAVYYCARSYYGDFDAYWGQGTLVTVSAAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 12 - protein, 215 aa, synthetic construct
  REGION 1..215: note = 27A6 light chain
QAVVTQESALTTSPGETVTLTCRSSIGAVTTSNHANWVQEKPDHLFTGLIGGTNNRAPGVPARFSGSLIGDKAVLTITGAQTEDEAIYFCALWYSNHWVFGGGTKLTVLGQPKSSPSVTLFPPSSEELETNKATLVCTITDFYPGVVTVDWKVDGTPVTQGMETTQPSKQSNNKYMASSYLTLTARAWERHSSYSCQVTHEGHTVEKSLSRADCS

(19) United States
(12) Patent Application Publication
(10) Pub. No.: US 2025/0084173 A1
(43) Pub. Date: Mar. 13, 2025

(54) TrkA ANTIBODY AND APPLICATION THEREOF

(71) Applicants: 4B TECHNOLOGIES (SUZHOU) LIMITED, Suzhou, Jiangsu (CN); 4B TECHNOLOGIES (BEIJING) CO., LIMITED, Beijing (CN)

(72) Inventors: Bai LU, Suzhou, Jiangsu (CN); Xiaoming GUAN, Jiangsu (CN)

(73) Assignees: 4B TECHNOLOGIES (SUZHOU) LIMITED, Suzhou, Jiangsu (CN); 4B TECHNOLOGIES (BEIJING) CO., LIMITED, Beijing (CN)

(21) Appl. No.: 18/724,809

(22) PCT Filed: Dec. 27, 2022
(86) PCT No.: PCT/CN2022/142200

§ 371 Date: Jun. 27, 2024

(30) Foreign Application Priority Data
Dec. 28, 2021 (WO) PCT/CN2021/142017

(51) Int. Cl. C07K 16/28; A61K 39/00; A61P 27/00

(52) U.S. Cl. CPC C07K 16/2863; A61P 27/00; A61K 2039/505; C07K 2317/24; C07K 2317/33; C07K 2317/76; C07K 2317/92

(57) ABSTRACT

The present disclosure provides TrkA antibodies, compositions comprising such antibodies, and methods of using such antibodies for the prevention and/or treatment of a disease or disorder associated with an inappropriate expression or function of TrkA, such as pain.

### BACKGROUND OF THE INVENTION

Nerve growth factor (NGF) acts through two membrane receptors. One is the relatively low affinity p75 receptor. The other is a 140 KDa high affinity receptor, known as TrkA.

NGF has potential use in the treatment of a wide range of disorders, such as various neurodegenerative disorders (including Alzheimer's disease), diabetes and leprosy.

Numerous evidence indicates the system constituted by the nerve growth factor (NGF) and its high-affinity receptor TrkA as a possible target for pain therapy. For this reason, antibodies capable of neutralizing the biological activity of the NGF/TrkA signaling by blocking the TrkA receptor may represent an important opportunity for pain therapy, in particular for chronic pain.

### SUMMARY OF THE INVENTION

The present disclosure provides a TrkA antibody and the application thereof.

In one aspect, the present disclosure provides an antibody or an antigen fragment thereof, which exhibits one of more of the following properties: 1) is capable of specifically binding to TrkA; 2) is capable of competing with NGF for binding to TrkA; 3) is capable of activating TrkA signaling; 4) is capable of inducing membrane TrkA internalization; 5) is capable of mitigating CFA-induced pain sensitization; and 6) is capable of inhibiting NGF-dependent signaling.

In some embodiments, the antibody is selected from the group consisting of: a monoclonal antibody, a chimeric antibody, a humanized antibody, a fully human antibody and a bispecific antibody.

In some embodiments, the antigen binding fragment is selected from the group consisting of: a Fab fragment, a Fab′ fragment, a F(ab)₂ fragment, a Fv fragment, a ScFv and a VHH.

In some embodiments, the antibody or the antigen binding fragment thereof comprises at least one of heavy chain CDRs 1-3 of a heavy chain variable region, and said heavy chain variable region comprises an amino acid sequence as set forth in SEQ ID NO: 7.

In some embodiments, the antibody or the antigen binding fragment thereof comprises a heavy chain CDR1 (HCDR1), and said heavy chain CDR1 comprises an amino acid sequence as set forth in SEQ ID NO: 1.

In some embodiments, the antibody or the antigen binding fragment thereof comprises a heavy chain CDR2 (HCDR2), and said heavy chain CDR2 comprises an amino acid sequence as set forth in SEQ ID NO: 2.

In some embodiments, the antibody or the antigen binding fragment thereof comprises a heavy chain CDR3 (HCDR3), and said heavy chain CDR3 comprises an amino acid sequence as set forth in SEQ ID NO: 3.

In some embodiments, the antibody or the antigen binding fragment thereof comprises a heavy chain variable region (VH), and said heavy chain variable region comprises an amino acid sequence as set forth in SEQ ID NO: 7.

In some embodiments, the antibody or the antigen binding fragment thereof comprises a heavy chain constant region.

In some embodiments, the heavy chain constant region comprises an amino acid sequence as set forth in SEQ ID NO: 9.

In some embodiments, the antibody or the antigen binding fragment thereof comprises at least one of light chain CDRs 1-3 of a heavy chain variable region, and said light chain variable region comprises an amino acid sequence as set forth in SEQ ID NO: 8.

In some embodiments, the antibody or the antigen binding fragment thereof comprises a light chain CDR1 (LCDR1), and said light chain CDR1 comprises an amino acid sequence as set forth in SEQ ID NO: 4.

In some embodiments, the antibody or the antigen binding fragment thereof comprises a light chain CDR2 (LCDR2), and said light chain CDR2 comprises an amino acid sequence as set forth in SEQ ID NO: 5.

In some embodiments, the antibody or the antigen binding fragment thereof comprises a light chain CDR3 (LCDR3), and said light chain CDR3 comprises an amino acid sequence as set forth in SEQ ID NO: 6.

In some embodiments, the antibody or the antigen binding fragment thereof comprises a light chain variable region, and said light chain variable region comprises an amino acid sequence as set forth in SEQ ID NO: 8.

In some embodiments, the antibody or the antigen binding fragment thereof comprises a light chain constant region.

In some embodiments, the light chain constant region comprises an amino acid sequence as set forth in SEQ ID NO: 10.

In some embodiments, the antibody or the antigen binding fragment thereof comprises heavy chain CDRs1-3 and light chain CDRs1-3, said heavy chain CDR1 comprises an amino acid sequence as set forth in SEQ ID NO: 1, said heavy chain CDR2 comprises an amino acid sequence as set forth in SEQ ID NO: 2, said heavy chain CDR3 comprises an amino acid sequence as set forth in SEQ ID NO: 3, said light chain CDR1 comprises an amino acid sequence as set forth in SEQ ID NO: 4, said light chain CDR2 comprises an amino acid sequence as set forth in SEQ ID NO: 5, said light chain CDR3 comprises an amino acid sequence as set forth in SEQ ID NO: 6.

In some embodiments, the antibody or the antigen binding fragment thereof comprises a heavy chain, said heavy chain comprises an amino acid sequence as set forth in SEQ ID NO: 11.

In some embodiments, the antibody or the antigen binding fragment thereof comprises a light chain, said light chain comprises an amino acid sequence as set forth in SEQ ID NO: 12.

In another aspect, the present application provides a fusion protein comprising the antibody or the antigen binding fragment thereof of the present application.

In another aspect the present application provides a protein conjugate, comprising the antibody or the antigen binding fragment thereof of the present application, or the fusion protein of the present application.

In another aspect, the present application provides an isolated nucleic acid molecule or molecules, encoding for the antibody or the antigen binding fragment thereof of the present application, or the fusion protein of the present application.

In another aspect, the present application provides a vector or vectors, comprising the isolated nucleic acid molecule or molecules of the present application.

In another aspect, the present application provides a cell, comprising the isolated nucleic acid molecule or molecules of the present application, or the vector or vectors of the present application.

In another aspect, the present application provides a composition, comprising the antibody or the antigen binding fragment thereof of the present application, the fusion protein of the present application, the protein conjugate of the present application, the isolated nucleic acid molecule or molecules of the present application, the vector or vectors of the present application, and/or the cell of the present application, and optionally a pharmaceutically acceptable excipient.

In another aspect, the present application provides a use of the antibody or the antigen binding fragment thereof of the present application, the fusion protein of the present application, the protein conjugate of the present application, the isolated nucleic acid molecule or molecules of the present application, the vector or vectors of the present application, and/or the cell of the present application in the manufacture of a medicament for preventing and/or treating a disease or disorder associated with an inappropriate expression or function of TrkA and/or TrkA/NGF signaling pathway.

In some embodiments, the disease or disorder comprises pain.

In some embodiments, the pain comprises chronic pain.

In another aspect, the present application provides a use of the antibody or the antigen binding fragment thereof of the present application, or the fusion protein of the present application in the manufacture of an agent for determining the presence and/or amount of TrkA and/or TrkA/NGF pathway in a sample.

In another aspect, the present application provides a method for producing the antibody or the antigen binding fragment thereof of the present application, or the fusion protein of the present application, comprising culturing the cell of the present application under conditions enabling expression of the antibody or the antigen binding fragment thereof of the present application, or the fusion protein of the present application.

In another aspect, the present application provides a method for preventing and/or treating a disease or disorder in a subject in need thereof, comprising administering to the subject an effective amount of the antibody or the antigen binding fragment thereof of the present application, the fusion protein of the present application, the protein conjugate of the present application, the isolated nucleic acid molecule or molecules of the present application, the vector or vectors of the present application, and/or the cell of the present application, wherein the disease or disorder is a disease or disorder associated with an inappropriate expression or function of TrkA and/or TrkA/NGF signaling pathway.

In some embodiments, the disease or disorder comprises pain.

In some embodiments, the pain comprises chronic pain.

In another aspect, the present application provides a method for determining the presence and/or amount of TrkA and/or TrkA/NGF pathway in a sample, comprising: a) contacting the sample with the antibody or the antigen binding fragment thereof of the present application, or the fusion protein of the present application; and b) determining the presence and/or amount of the antibody or the antigen binding fragment thereof, or the fusion protein bound to the sample.

In another aspect, the present application provides the antibody or the antigen binding fragment thereof of the present application, the fusion protein of the present application, the protein conjugate of the present application, the isolated nucleic acid molecule or molecules of the present application, the vector or vectors of the present application, or the cell of the present application, for a) preventing and/or treating a disease or disorder, and/or b) determining the presence and/or amount of TrkA in a sample, wherein the disease or disorder is a disease or disorder associated with an inappropriate expression or function of TrkA and/or TrkA/NGF signaling pathway.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWING

The novel features of the invention are set forth with particularity in the appended claims. A better understanding of the features and advantages of the present invention will be obtained by reference to the following detailed description that sets forth illustrative embodiments, in which the principles of the invention are employed, and the accompanying drawings (also “figure” and “FIG.” herein), of which:

FIG. 1a-1f illustrate the identification and characterization of the TrkA antibody of the present application.

FIG. 2a-2b illustrates the effects of the TrkA antibody of the present application on PC12 outgrowth.

FIG. 3 illustrates that the TrkA antibody maintains TrkA signaling activation in PC12 cells.

FIG. 4a-4e illustrate the time course of TrkA signaling activation by the TrkA antibody of the present application.

FIG. 5a-5e illustrate that the TrkA antibody of the present application mitigates CFA-induced pain sensitization.

FIG. 6a-6f illustrate that the TrkA antibody of the present application induces membrane TrkA internalization and degradation.

FIG. 7a-7g illustrate that the TrkA antibody of the present application inhibits NGF-dependent signaling.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The term “antibody”, as used herein, generally refers to an immunoglobulin or an immunoglobulin-like molecule capable of specifically recognizing or binding to an antigen. An antibody may comprise a light chain (L) and a heavy chain (H). The light chains of an antibody can be classified as κ and λ light chains. The heavy chains can be classified as μ, δ, γ, α or ε, and the isotypes of an antibody are defined as IgM, IgD, IgG (e.g., IgG1, IgG2, IgG3 or IgG4 subtype), IgA and IgE, respectively. Each heavy chain may comprise a heavy chain variable region (VH) and a heavy chain constant region (CH). The heavy chain constant region may comprise three domains (CH1, CH2 and CH3). Each light chain may comprise a light chain variable region (VL) and a light chain constant region (CL). The light chain constant region may comprise a CL domain. The VH and VL regions can also be subdivided into regions with high variability known as complementarity determining regions (CDRs) interspersed with more conserved regions known as framework regions (FRs). Each VH and VL consists of 3 CDRs and 4 FRs arranged from N-terminal to C-terminal in the following order: FR1, CDR1, FR2, CDR2, FR3, CDR3, FR4. The variable regions (VH and VL) of each heavy/light chain pair form the antibody binding site, respectively. Distribution of amino acids to regions or domains follows the definition of Kabat Sequences of Proteins of Immunological Interest (National Institutes of Health, Bethesda, Md. (1987 and 1991)), or Chothia & Lesk (1987) J. Mol. Biol. 196:901-917; Chothia et al. (1989) Nature 342:878-883. The term “antibody” is not limited by any antibody-producing method. For example, it includes recombinant antibodies, monoclonal antibodies, and other forms of antibodies. In some cases, an antibody of the present disclosure is an isolated antibody.

The term “antigen binding fragment”, as used herein, generally refers to one or more fragments of a full-length antibody that retain the ability to bind the same antigen to which the antibody binds (e.g., TrkA) and/or competes against an intact antibody for an antigen-specific binding. Antigen binding fragment can be produced by recombinant DNA techniques or by enzymatic or chemical cleavage of intact antibodies. In some cases, the antigen binding fragment includes Fab, Fab′, F(ab′)₂, F(ab)₂, VHH, Fd, Fv, dAb and complementarity determining region (CDR) fragments, single chain antibodies (e.g., ScFv), chimeric antibodies, diabodies, and polypeptides that comprise at least a portion of an antibody that is sufficient to confer specific antigen-binding ability to the polypeptide.

The term “TrkA”, as used herein, generally refers to high affinity nerve growth factor receptor or neurotrophic tyrosine kinase receptor type 1 or TRK1-transforming tyrosine kinase protein or Tropomyosin-related kinase A or Tyrosine kinase receptor or Tyrosine kinase receptor A or Trk-A or gp140trk or p140-TrkA or MTC or TRK. TrkA is a receptor tyrosine kinase involved in the development and the maturation of the central and peripheral nervous systems through regulation of proliferation, differentiation, and survival of sympathetic and nervous neurons. TrkA is the high affinity receptor for NGF which is its primary ligand; it may also bind and be activated by NTF3/neurotrophin-3. TrkA as used herein may comprise functional fragments, variants, isoforms, and species homologs of human TrkA. Accordingly, antibodies of this disclosure may, in certain cases, cross-react with TrkA from species other than human. In certain embodiments, the antibodies may be completely specific for one or more human TrkA proteins and may not exhibit species or other types of non-human cross-reactivity. The complete amino acid sequence of the four known human TrkA isoforms are found under the UniProt/Swiss-Prot accession number P04629 (Consortium T U, (2012) Nucleic Acids Res. 40 (D1): D71-D5). The four isoforms are produced by alternative splicing: isoform TrkA-I is found in most non-neuronal tissues (UniProt/Swiss-Prot accession number P04629-2), while isoform TrkA-II is primarily expressed in neuronal cells (UniProt/Swiss-Prot accession number P04629-1), and isoform TrkA-III is specifically expressed by pluripotent neural stem and neural crest progenitors (UniProt/Swiss-Prot accession number P04629-4). A fourth isoform which differs from isoform TrkA-II at residues 1-71 and lacks residues 393 to 398 is known as isoform 3 (UniProt/Swiss-Prot accession number P04629-3). TrkA-II isoform is the major known isoform of TrkA. Isoform TrkA-I has enhanced responsiveness to NTF3 neurotrophin whereas isoform TrkA-III is constitutively active and does not bind NGF.

The term “biological activities of TrkA” or “TrkA biological activities”, as used herein, generally refers to any one or more of the following: the ability to bind NGF or other neurotrophins; the ability to activate an NGF induced signaling pathway; the ability to promote cell differentiation, proliferation, survival, growth, migration and other changes in cell physiology, including (in the case of neurons, including peripheral and central neuron) change in neuronal morphology, synaptogenesis, synaptic function, neurotransmitter and/or neuropeptide release and regeneration following damage; and the ability to mediate pain and cancer pain associated with bone metastasis.

The term “TrkA/NGF signaling pathway”, as used herein, generally refers to the signaling pathway relating to Nerve Growth Factor (NGF) and Tyrosine Kinase A (TrkA). The NGF peptides may engage the NGF pathway through TrkA phosphorylation, and activation of ShcC/PI3K and Plc-Y/MAPK signaling, promoting AKT-dependent survival and CREB-driven neuronal activity. The TrkA/NGF signaling pathway may promote survival and innervation of sympathetic and sensory neurons, for example, may relate to the endocytosis and retrograde transport of NGF/TrkA-containing endosomes from the axon terminal to the cell body for activation of NGF-inducible gene expression responsible for neuronal survival and development.

The term “binding specificity”, as used herein, generally refers to an ability of one substance to bind another substance specifically, and not substantially bind to any other substance at random. For example, one protein may bind to another protein specifically due to their specific structures. Binding specificity may be measured by, e.g., cross-competing assays or other binding assays known in the art.

The term “K_{D}”, as used herein, generally refers to the dissociation constant, a specific type of equilibrium constant that measures the propensity of a larger object to separate (dissociate) reversibly into smaller components, as when a complex falls apart into its component molecules. The dissociation constant is the inverse of the association constant. In the specific case of antibodies (Ab) binding to antigen (Ag), usually the term affinity constant refers to the association constant.

The term “monoclonal antibody”, as used herein, generally refers to antibodies that are made by identical immune cells that are all clones of a unique parent cell. Monoclonal antibodies can have a monovalent affinity, in that they bind to the same epitope (the part of an antigen that is recognized by the antibody). Sometimes, monoclonal antibodies may also be multi specific, such as bispecific or tri specific. It has become an important tool in biochemistry, molecular biology, and medicine. Several monoclonal antibody technologies had been developed recently, such as phage display, single B cell culture, single cell amplification from various B cell populations and single plasma cell interrogation technologies.

The term “chimeric antibody”, as used herein, generally refers to an antibody in which the Variable (V) region of light and heavy chains is of mouse origin, while the Constant (C) region is of human origin. In general, the chimeric antibody may retain the specificity and affinity of the original mouse monoclonal antibody, but HAMA response may be significantly reduced.

The term “humanized antibody”, as used herein, generally refers to antibodies from non-human species whose protein sequences have been modified to increase their similarity to antibody variants produced naturally in humans. The amino acid sequence of a humanized antibody may be essentially identical to that of a human variant, despite the non-human origin of some of its complementarity determining region (CDR) segments responsible for the ability of the antibody to bind to its target antigen.

The term “fully human antibody” and “human antibody” are used interchangeably herein, and generally refers to an antibody that comprises a human variable region and, most preferably a human constant region. In specific embodiments, the terms refer to an antibody that comprises a variable region and constant region of human origin. The term “fully human antibody” includes antibodies having variable and constant regions corresponding to human germline immunoglobulin sequences as described by Kabat et al. (See Kabat et al. (1991) Sequences of Proteins of Immunological Interest, Fifth Edition, U.S. Department of Health and Human Services, NIH Publication No. 91-3242).

The term “Fab fragment”, as used herein, generally refers to a portion (such as an antigen-binding fragment) of an immunoglobulin molecule. A Fab fragment may comprise one light chain and part of a heavy chain with a single antigen-binding site. A Fab fragment may be obtained by papain digestion of an immunoglobulin molecule. For example, a Fab fragment may be composed of one constant and one variable domain of each of the heavy and the light chain. The variable domain may contain the paratope (the antigen-binding site), comprising a set of the complementarity determining regions, at the amino-terminal end of the immunoglobulin molecule. The enzyme papain may be used to cleave an immunoglobulin molecule into two Fab fragments and one Fc fragment. The enzyme pepsin cleaves below the hinge region, so a F(ab′)₂ fragment and a pFc′ fragment is formed. Divalent F(ab)₂ or F(ab′)₂ fragments have two antigen binding regions that are linked by disulfide bonds. Reduction of F(ab)₂ or F(ab′)₂ fragments produce 2 monovalent Fab or Fab′ fragments, which have a free sulfhydryl group that is useful for conjugation to other molecules.

The term “Fv fragment”, as used herein, generally refers to the smallest fragment made from enzymatic cleavage of IgG and IgM class antibodies. Fv fragments have the antigen-binding site made of the VH and VL regions, but they lack the CH1 and CL regions. The VH and VL chains may be held together in Fv fragments by non-covalent interactions.

The term “ScFv”, as used herein, generally refers to a single-chain antibody fragment. An ScFv may be a recombinant single chain polypeptide molecule in which light and heavy chain variable regions of an antibody are connected, either directly or via a peptide linker. Single chain antibodies (ScFv) generally do not include portions of the Fc region of antibody, although methods are known for adding such regions to known ScFv molecules if desired. See Helfrich et al., A rapid and versatile method for harnessing ScFv antibody fragments with various biological functions. J Immunol Methods 237:131-145 (2000) and de Haard et al., Creating and engineering human antibodies for immunotherapy. Advanced Drug Delivery Reviews 31:5-31 (1998).

The term “fusion protein”, as used herein, generally refers to a polypeptide that comprises, or alternatively consists of, an amino acid sequence of a polypeptide fused directly or indirectly (e.g., via a linker) to an amino acid sequence of a heterologous polypeptide (i.e., a polypeptide of a different origin, sequence or structure).

The term “protein conjugate”, as used herein, generally refers to a conjugate comprising a protein (e.g., an antibody or a functional fragment thereof) conjugated to one or more additional moieties, such as cytotoxic agents, e.g., a chemotherapeutic agent, a drug, a growth inhibitory agent, a toxin (e.g., a protein toxin, an enzymatically active toxin of bacterial, fungal, plant, or animal origin, or fragments thereof), a label (e.g., a fluorescent label) and/or a radioactive isotope (i.e., a radioconjugate).

A number of CDR definitions are in use and are encompassed herein. The Kabat definition is based on sequence variability and is the most commonly used (Kabat E A et al., ibid.). Chothia refers instead to the location of the structural loops (Chothia & Lesk J. (1987) Mol. Biol. 196:901-917). The AbM definition is a compromise between the Kabat and the Chothia definitions and is used by Oxford Molecular's AbM antibody modelling software (Martin A C R et al., (1989) PNAS USA 86:9268-9272; Martin A C R et al., (1991) Methods Enzymol. 203:121-153; Pedersen J T et al., (1992) Immunomethods 1:126-136; Rees A R et al., (1996) In Sternberg M. J. E. (ed.), Protein Structure Prediction. Oxford University Press, Oxford, 141-172). The contact definition has been recently introduced (MacCallum R M et al., (1996) J. Mol. Biol. 262:732-745) and is based on an analysis of the available complex structures available in the Protein Databank. The definition of the CDR by IMGT®, the international ImMunoGeneTics information System® (http://www.imgt.org) is based on the IMGT numbering for all immunoglobulin and T cell receptor V-REGIONs of all species (IMGT®, the international ImMunoGeneTics information System®; Lefranc M P et al., (1999) Nucleic Acids Res. 27 (1): 209-12; Ruiz M et al., (2000) Nucleic Acids Res. 28 (1): 219-21; Lefranc M P (2001) Nucleic Acids Res. 29 (1): 207-9; Lefranc M P (2003) Nucleic Acids Res. 31 (1): 307-10; Lefranc M P et al., (2005) Dev. Comp. Immunol. 29 (3): 185-203; Kaas Q et al., (2007) Briefings in Functional Genomics & Proteomics, 6 (4): 253-64).

The term “isolated nucleic acid molecule or molecules” as used herein, generally refers to a polymeric form of nucleotides of any length, either deoxyribonucleotides or ribonucleotides, or analogs thereof, isolated from its native environment, or that is artificially synthesized.

The term “vector or vectors” as used herein, generally refers to a nucleic acid vehicle into which a polynucleotide encoding a protein can be inserted and expressed. The genetic material elements carried in the vector can be expressed in a host cell by transforming, transducing, or transfecting the host cell with the vector. A vector may contain a variety of elements that control expression, including promoter sequences, transcriptional initiation sequences, enhancer sequences, selection elements, and reporter genes. In addition, the vector may also contain an origin of replication. It is also possible that the vector may include components that assist its entry into the cell, such as viral particles, liposomes or protein shells, but not only these substances.

The term “cell” as used herein, generally refers to a cell that may be used to carry the vector or vectors of the present disclosure, or be used to express or produce the antibody, the antigen binding fragment of the present disclosure. A cell of the present disclosure may be a host cell.

The terms “disease” and “disorder” may be used interchangeably herein, and generally refer to any condition that impairs the normal functioning of the body. Disease is often construed as a medical condition associated with specific symptoms and signs. It may be caused by external factors such as pathogens or by internal dysfunctions, particularly of the immune system, such as an immunodeficiency, or by a hypersensitivity, including allergies and autoimmunity.

As used herein, the term “subject” includes any human or non-human animal. The term “non-human animal” includes all vertebrates, e.g., mammals and non-mammals, such as primates, sheep, dogs, cats, horses, cows, chickens, amphibians, reptiles, etc. For example, the subject may be human.

The term “effective amount”, as used herein, generally refers to a dose sufficient to provide concentrations high enough to impart a beneficial effect on the recipient thereof. The specific therapeutically effective dose level for any particular subject will depend upon a variety of factors including the disease or disorder being treated, the severity of the disease or disorder, the activity of the specific component, the route of administration, the rate of clearance, the duration of treatment, the age, body weight, sex, diet, and general health of the subject, and other related factors.

The term “pharmaceutically acceptable excipient”, as used herein, generally refers to any and all solvents, dispersion media, coatings, isotonic and absorption delaying agents, etc., that are compatible with pharmaceutical administration.

The term “about”, as used herein, generally refers to an approximation to a given value that would reasonably be inferred based on the ordinary skill in the art, including equivalents and approximations due to the experimental and/or measurement conditions for such given value. For example, it may refer to a value that is no more than 10% above or below the value being modified by the term.

The terms “polypeptide” or “protein”, as used herein, generally refers to macromolecule having the amino acid sequence of a native protein, that is, a protein produced by a naturally-occurring and non-recombinant cell; or it is produced by a genetically-engineered or recombinant cell, and comprise molecules having the amino acid sequence of the native protein, or molecules having deletions from, additions to, and/or substitutions of one or more amino acids of the native sequence. The term also includes amino acid polymers in which one or more amino acids are chemical analogs of a corresponding naturally occurring amino acid and polymers. The terms “polypeptide” and “protein” specifically encompass TrkA antigen binding proteins, antibodies, or sequences that have deletions from, additions to, and/or substitutions of one or more amino acid of antigen-binding protein. The term “polypeptide fragment” refers to a polypeptide that has an amino-terminal deletion, a carboxyl-terminal deletion, and/or an internal deletion as compared with the full-length native protein. Such fragments can also contain modified amino acids as compared with the native protein. In certain embodiments, fragments are about five to 500 amino acids long. For example, fragments can be at least 5, 6, 8, 10, 14, 20, 50, 70, 100, 110, 150, 200, 250, 300, 350, 400, or 450 amino acids long. Useful polypeptide fragments include immunologically functional fragments of antibodies, including binding domains. In the case of a TrkA-binding antibody, useful fragments include but are not limited to a CDR region, a variable domain of a heavy and/or light chain, a portion of an antibody chain or just its variable region including two CDRs, and the like.

The term “isolated protein” (such as isolated antibody), as used herein, generally refers to a subject protein (1) is free of at least some other proteins with which it would normally be found, (2) is essentially free of other proteins from the same source, e.g., from the same species, (3) is expressed by a cell from a different species, (4) has been separated from at least about 50 percent of polynucleotides, lipids, carbohydrates, or other materials with which it is associated in nature, (5) is operably associated (by covalent or noncovalent interaction) with a polypeptide with which it is not associated in nature, or (6) does not occur in nature. Typically, an “isolated protein” constitutes at least about 5%, at least about 10%, at least about 25%, or at least about 50% of a given sample. Genomic DNA, cDNA, mRNA or other RNA, of synthetic origin, or any combination thereof can encode such an isolated protein. Preferably, the isolated protein is substantially free from proteins or polypeptides or other contaminants that are found in its natural environment that would interfere with its therapeutic, diagnostic, prophylactic, research or other use.

A “variant” of a polypeptide (e.g., an antigen binding protein, or an antibody) comprises an amino acid sequence wherein one or more amino acid residues are inserted into, deleted from and/or substituted into the amino acid sequence relative to another polypeptide sequence. Variants include fusion proteins.

Unless otherwise indicated, the term “antibody” includes, in addition to antibodies comprising two full-length heavy chains and two full-length light chains, derivatives, variants, fragments, and muteins thereof, examples of which are described below. Furthermore, unless explicitly excluded, antibodies include monoclonal antibodies, bispecific antibodies, minibodies, domain antibodies, synthetic antibodies (sometimes referred to herein as “antibody mimetics”), chimeric antibodies, humanized antibodies, human antibodies, antibody fusions (sometimes referred to herein as “antibody conjugates”), and fragments thereof, respectively. In some embodiments, the term also encompasses peptibodies.

As used herein, “substantially” or “substantial” generally means to a great or significant extent (e.g., to an extent of at least 50%, at least 60%, at least 70%, at least 80%, at least 85%, at least 90%, at least 95%, or at least 99% or more).

### Antibody or an Antigen Binding Fragment Thereof

In one aspect, the present disclosure provides an antibody or an antigen binding fragment thereof, which binds to TrkA.

The antibody, or the antigen binding fragment thereof may specifically bind to TrkA, and does not substantially bind to Tropomyosin receptor kinase B (TrkB), Tropomyosin receptor kinase C (TrkC), or p75 receptor.

The antibody, or the antigen binding fragment thereof may be capable of competing with NGF for binding to TrkA.

The antibody, or the antigen binding fragment thereof may be capable of activating TrkA signaling.

The antibody, or the antigen binding fragment thereof may be capable of inducing membrane TrkA internalization. In some embodiments, the antibody or the antigen binding fragment thereof may promote membrane TrkA internalization and degradation in cell lines as well as dorsal root ganglion (DRG) neurons.

The antibody, or the antigen binding fragment thereof may be capable of mitigating CFA-induced pain sensitization.

The antibody, or the antigen binding fragment thereof may be capable of inhibiting NGF-dependent signaling.

The antibody, or the antigen binding fragment may be capable of inhibiting the function of NGF in PC12 cells neurons.

In some cases, the antibody may be selected from the group consisting of: a monoclonal antibody, a chimeric antibody, a humanized antibody, a fully human antibody and a bispecific antibody.

In some cases, the antigen binding fragment may be selected from the group consisting of: a Fab fragment, a Fab′ fragment, a F(ab)₂ fragment, a Fv fragment, a ScFv and a VHH.

TrkA also known as high affinity nerve growth factor receptor, neurotrophic tyrosine kinase receptor type 1, or TRK1-transforming tyrosine kinase protein is a protein that in humans is encoded by the NTRK1 gene. In some cases, the TrkA may be human TrkA, a mouse TrkA and/or a rat TrkA. For example, human TrkA may be identified with Accession No. P04629 in the UniProtKB database, or may be a variant thereof, e.g., having a sequence identity of at least 80% (e.g., at least 85%, at least 90%, at least 91%, at least 92%, at least 93%, at least 94%, at least 95%, at least 96%, at least 97%, at least 98%, at least 99% or higher) thereto.

In some embodiments, the antibody, or the antigen binding fragment thereof comprises one or more CDRs (e.g., 1, 2, 3, 4, 5 or 6 CDRs). In some embodiments, the antibody, or the antigen binding fragment thereof comprises (a) a polypeptide structure and (b) one or more CDRs that are inserted into and/or joined to the polypeptide structure. The polypeptide structure can take a variety of different forms. For example, it can be, or comprise, the framework of a naturally occurring antibody, or fragment or variant thereof, or can be completely synthetic in nature.

In certain embodiments, the polypeptide structure of the antibody, or the antigen binding fragment thereof is an antibody or is derived from an antibody, including, but not limited to, monoclonal antibodies, bispecific antibodies, minibodies, domain antibodies, synthetic antibodies (sometimes referred to herein as “antibody mimetics”), chimeric antibodies, humanized antibodies, antibody fusions (sometimes referred to as “antibody conjugates”), and portions or fragments of each, respectively. In some instances, the antibody, or the antigen binding fragment thereof is an immunological fragment of an antibody (e.g., a Fab, a Fab′, a F(ab′)₂, a scFv or a VHH).

The antibody, or the antigen binding fragment thereof may comprise a heavy chain constant region. The heavy chain constant region may comprise a IgG constant region (such as a human IgG1, IgG2, or IgG4 constant region). In some embodiments, the heavy chain constant region comprises an amino acid sequence as set forth in SEQ ID NO: 9.

Variable regions of immunoglobulin chains generally exhibit the same overall structure, comprising relatively conserved framework regions (FR) joined by three hypervariable regions, more often called “complementarity determining regions” or CDRs. The CDRs from the two chains of each heavy chain/light chain pair mentioned above typically are aligned by the framework regions to form a structure that binds specifically with a specific epitope on the target protein (e.g., TrkA). From N-terminal to C-terminal, naturally occurring light and heavy chain variable regions both typically conform with the following order of these elements: FR1, CDR1, FR2, CDR2, FR3, CDR3 and FR4. A numbering system has been devised for assigning numbers to amino acids that occupy positions in each of these domains. This numbering system is defined in Kabat Sequences of Proteins of Immunological Interest (1987 and 1991, NIH, Bethesda, MD), or Chothia & Lesk, 1987, J. MOL Biol. 196:901-917; Chothia et al., 1989, Nature 342:878-883.

In some embodiments, the set of variable regions can be attached to a heavy and light chain constant region to form a complete antibody heavy and light chain, respectively. Further, each of the so generated heavy and light chain sequences can be combined to form a complete antibody structure.

The antibody, or the antigen binding fragment thereof may comprise a light chain constant region. The light chain constant region may comprise a Igκ constant region or a Igλ constant region. In some embodiments, the light chain constant region comprises an amino acid sequence as set forth in SEQ ID NO: 10.

In some embodiments, the antibody or the antigen binding fragment thereof comprises at least one of heavy chain CDRs1-3 of a heavy chain variable region, and the heavy chain variable region comprises an amino acid sequence as set forth in any one of SEQ ID NO: 7.

In some embodiments, the antibody or the antigen binding fragment thereof comprises a heavy chain CDR1, and said heavy chain CDR1 comprises an amino acid sequence as set forth in SEQ ID NO: 1.

In some embodiments, the antibody or the antigen binding fragment thereof comprises heavy chain CDR2, and said heavy chain CDR2 comprises an amino acid sequence as set forth in SEQ ID NO: 2.

In some embodiments, the antibody or the antigen binding fragment thereof comprises heavy chain CDR3, and said heavy chain CDR3 comprises an amino acid sequence as set forth in SEQ ID NO: 3.

In some embodiments, the antibody or the antigen binding fragment thereof comprises heavy chain CDR1-3, wherein the heavy chain CDR1 comprises an amino acid sequence as set forth in SEQ ID NO: 1, the heavy chain CDR2 comprises an amino acid sequence as set forth in SEQ ID NO: 2, and the heavy chain CDR3 comprises an amino acid sequence as set forth in SEQ ID NO: 3.

In some embodiments, the antibody or the antigen binding fragment thereof comprises a heavy chain variable region, and the heavy chain variable region comprises an amino acid sequence as set forth in SEQ ID NO: 7.

In some embodiments, the antibody or the antigen binding fragment thereof comprises at least one of light chain CDRs 1-3 of a light chain variable region, and said light chain variable region comprises an amino acid sequence as set forth in SEQ ID NO: 8.

In some embodiments, the antibody or the antigen binding fragment thereof comprises light chain CDR1, and said light chain CDR1 comprises an amino acid sequence as set forth in SEQ ID NO: 4.

In some embodiments, the antibody or the antigen binding fragment thereof comprises light chain CDR2, and said light chain CDR2 comprises an amino acid sequence as set forth in SEQ ID NO: 5

In some embodiments, the antibody or the antigen binding fragment thereof comprises light chain CDR3, and said light chain CDR3 comprises an amino acid sequence as set forth in SEQ ID NO: 6

In some embodiments, the antibody or the antigen binding fragment thereof comprises light chain CDR1-3, wherein the light chain CDR1 comprises an amino acid sequence as set forth in SEQ ID NO: 4, the light chain CDR2 comprises an amino acid sequence as set forth in SEQ ID NO: 5, and the light chain CDR3 comprises an amino acid sequence as set forth in SEQ ID NO: 6.

In some embodiments, the antibody or the antigen binding fragment thereof comprises a light chain variable region, and the light chain variable region comprises an amino acid sequence as set forth in SEQ ID NO: 8.

In some embodiment, the antibody or the antigen binding fragment thereof comprises a heavy chain, and the heavy chain comprises an amino acid sequence as set forth in SEQ ID NO: 11.

In some embodiment, the antibody or the antigen binding fragment thereof comprises a light chain, and the light chain comprises an amino acid sequence as set forth in SEQ ID NO: 12.

The antibody or antigen binding fragment may also encompass a homology or a variant thereof having substantially the same function/property thereto. In some cases, the homology or variant may be a polypeptide different from the antibody or antigen binding fragment thereof at least one amino acid. For example, the homology or variant may be a polypeptide different from the antibody or antigen binding fragment thereof by an addition, deletion or substitution of one or more amino acid, such as 1-50, 1-40, 1-30, 1-20, 1-15, 1-14, 1-13, 1-12, 1-11, 1-10, 1-9, 1-8, 1-7, 1-6, 1-5, 1-4, 1-3, or 1-2 amino acids. In some cases, the homology or variant may be a polypeptide having a sequence identity of at least 80% with the antibody or antigen binding fragment thereof. For example, the homology or variant may be a polypeptide having a sequence identity of 80% (e.g., at least 85%, at least 90%, at least 91%, at least 92%, at least 93%, at least 94%, at least 95%, at least 96%, at least 97%, at least 98%, at least 99% or higher) to the antibody or antigen binding fragment thereof.

The term “percent (%) sequence identity,” as used in the context of polypeptide sequences identified herein, generally refers to the percentage of amino acid residues or nucleotides in a query sequence that are identical with the amino acid residues or nucleotides of a second, reference polypeptide sequence or a portion thereof, after aligning the sequences and introducing gaps, if necessary, to achieve the maximum percent sequence identity, and not considering any conservative substitutions as part of the sequence identity. Alignment for purposes of determining percent amino acid/nucleotide sequence identity can be achieved in various ways that are within the skill in the art, for instance, using publicly available computer software such as BLAST, BLAST-2, ALIGN, NEEDLE or Megalign (DNASTAR) software. Those skilled in the art can determine appropriate parameters for measuring alignment, including any algorithms needed to achieve maximal alignment over the full length of the sequences being compared. Percent identity may be measured over the length of an entire defined polypeptide/polynucleotide sequence, or may be measured over a shorter length, for example, over the length of a fragment taken from a larger, defined polypeptide/polynucleotide sequence. It is understood that any fragment length supported by the sequences shown herein, in the tables, figures or Sequence Listing, may be used to describe a length over which percentage identity may be measured.

In the present application, the CDR sequences may be classified according to a specific CDR classification standard (for example, the Kabat method). It should be noted that if the amino acid sequence of a TrkA antibody or the antigen binding fragment thereof divided according to a specific CDR division standard (such as the Kabat method) is the same as the CDR sequence of the present application, then the TrkA antibody or the antigen binding fragment thereof is also within the protection scope of the present application. According to different CDR classification standards, the precise identification of CDR positions may be slightly different, and therefore the present application comprises not only the CDRs shown in the sequence listing but also the CDRs comprising in the VH and VL domains using other numbering systems.

In another aspect, the present application provides a fusion protein, comprising the antibody or the antigen binding fragment thereof of the present disclosure.

In another aspect, the present application provides a protein conjugate (such as an immunoconjugate), comprising the antibody or the antigen binding fragment thereof of the present disclosure.

### Nucleic Acid, Vector, Cell and Preparation Method

In another aspect, the present disclosure provides isolated nucleic acid or molecules, encoding for the antibody or the antigen binding fragment thereof, or the fusion protein.

The isolated nucleic acids may comprise one or more nucleic acid molecules, with each encoding for at least a part of the antibody of the present disclosure or an antigen binding fragment thereof. For example, the isolated nucleic acids may comprise at least two nucleic acid molecules, with one encoding for the antibody heavy chain or a fragment thereof, and one encoding for the antibody light chain or a fragment thereof. In some cases, the isolated nucleic acids may encode for a fusion protein.

The isolated nucleic acid or isolated nucleic acids may be synthesized using recombinant techniques well known in the art. For example, the isolated nucleic acid or isolated nucleic acids may be synthesized with an automated DNA synthesizer. Standard recombinant DNA and molecular cloning techniques include those described by Sambrook, J., Fritsch, E. F. and Maniatis, T. Molecular Cloning: A Laboratory Manual; Cold Spring Harbor Laboratory Press: Cold Spring Harbor, (1989) (Maniatis) and by T. J. Silhavy, M. L. Bennan, and L. W. Enquist, Experiments with Gene Fusions, Cold Spring Harbor Laboratory, Cold Spring Harbor, N.Y. (1984) and by Ausubel, F. M. et al., Current Protocols in Molecular Biology, pub. by Greene Publishing Assoc. and Wiley-Interscience (1987). Briefly, the subject nucleic acids may be prepared from genomic DNA fragments, cDNAs, and RNAs, all of which may be extracted directly from a cell or recombinantly produced by various amplification processes including but not limited to PCR and RT-PCR.

Direct chemical synthesis of nucleic acids typically involves sequential addition of 3′-blocked and 5′-blocked nucleotide monomers to the terminal 5′-hydroxyl group of a growing nucleotide polymer chain, wherein each addition is affected by nucleophilic attack of the terminal 5′-hydroxyl group of the growing chain on the 3′-position of the added monomer, which is typically a phosphorus derivative, such as a phosphotriester, phosphoramidite, or the like. See for example, Matteuci et al., Tet. Lett. 521:719 (1980); U.S. Pat. No. 4,500,707 to Caruthers et al.; and U.S. Pat. Nos. 5,436,327 and 5,700,637 to Southern et al.

In another aspect, the present disclosure provides a vector or vectors, comprising the isolated nucleic acid molecule or molecules.

The vector may be any linear nucleic acids, plasmids, phagemids, cosmids, RNA vectors, viral vectors and the like. Non-limiting examples of a viral vector may include a retrovirus, an adenovirus and an adeno-associated virus. In some cases, the vector is an expression vector, e.g. a phage display vector.

An expression vector may be suitable for use in particular types of host cells and not others. For example, the expression vector can be introduced into the host organism, which is then monitored for viability and expression of any genes/polynucleotides contained in the vector.

The expression vector may also contain one or more selectable marker genes that, upon expression, confer one or more phenotypic traits useful for selecting or otherwise identifying host cells that carry the expression vector. Non-limiting examples of suitable selectable markers for eukaryotic cells include dihydrofolate reductase and neomycin resistance.

The subject vectors can be introduced into a host cell stably or transiently by a variety of established techniques. For example, one method involves a calcium chloride treatment wherein the expression vector is introduced via a calcium precipitate. Other salts, for example calcium phosphate, may also be used following a similar procedure. In addition, electroporation (that is, the application of current to increase the permeability of cells to nucleic acids) may be used. Other examples of transformation methods include microinjection, DEAE dextran mediated transformation, and heat shock in the presence of lithium acetate. Lipid complexes, liposomes, and dendrimers may also be employed to transfect the host cells.

In another aspect, the present disclosure provides a cell (e.g., an isolated cell, such as a host cell), comprising the isolated nucleic acid molecule or molecules of the present disclosure or the vector or vectors of the present disclosure.

The cell may express the antibody, or the antigen binding fragment thereof of the present disclosure, or the fusion protein of the present disclosure. The cell may be a eukaryotic cell or a prokaryotic cell. An appropriate cell may be transformed or transfected with the nucleic acid(s) or vector(s) of the present disclosure and utilized for the expression and/or secretion of the antibody, the antigen binding fragment thereof, or the fusion protein. For example, the cell may be E. coli cells, other bacterial host cells, yeast cells, or various higher eukaryotic cells.

In another aspect, the present disclosure provides a method for producing the antibody or the antigen binding fragment thereof, or the fusion protein of the present disclosure, comprising culturing the cell of the present disclosure under conditions enabling expression of the antibody, the antigen binding fragment thereof, or the fusion protein.

The method optionally may further comprise harvesting the antibody or the antigen binding fragment thereof, or the fusion protein of the present disclosure.

### Compositions

In another aspect, the present disclosure provides a composition, comprising the antibody or the antigen binding fragment thereof, the fusion protein, the protein conjugate, the isolated nucleic acid molecule or molecules, the vector or vectors, and/or the cell of the present disclosure, and optionally a pharmaceutically acceptable excipient.

In some cases, the composition further comprises an effective amount of an additional therapeutically active component, for example, an additional therapeutically active component for treating a disease or a disorder associated with an inappropriate expression or function of IL-17A. Each of the active components may be present in the pharmaceutical composition in a pharmaceutically active amount. In the composition, the antibody, the fragment thereof of the present application may or may not be associated with the additional active component.

Described below are non-limiting exemplary pharmaceutical compositions and methods for preparing the same. The pharmaceutical composition may, for example, be in a form suitable for oral administration as a tablet, capsule, pill, powder, sustained release formulations, solution, suspension, for parenteral injection as a sterile solution, suspension or emulsion, for topical administration as an ointment or cream or for rectal administration as a suppository. The pharmaceutical composition may be in unit dosage forms suitable for single administration of precise dosages. In some cases, the pharmaceutical composition may be a liquid pharmaceutical composition.

Pharmaceutical compositions of the disclosure can be presented as discrete dosage forms, with each dosage containing a predetermined amount of an active ingredient as a powder or in granules, a solution, or a suspension in an aqueous or non-aqueous liquid. Such dosage forms can be prepared by any of the methods known to a skilled person, for example, it may include the step of bringing the active ingredient into association with the carrier, which constitutes one or more other ingredients. In general, the compositions are prepared by uniformly and intimately mixing the active ingredient with liquid carriers or finely divided solid carriers or both, and then, if necessary, shaping the product into the desired presentation.

The antibody, the antigen binding fragment thereof, the fusion protein, or the protein conjugate of the present disclosure can be combined in an intimate admixture with a pharmaceutical carrier according to conventional pharmaceutical compounding techniques. The carrier can take a wide variety of forms depending on the form of preparation desired for administration.

The composition can further include one or more pharmaceutically acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and/or mixtures thereof.

The pharmaceutical compositions of the present disclosure may comprise a therapeutically effective amount of the active agent (e.g., the antibody, the antigen binding fragment thereof, or the fusion protein of the present disclosure). A therapeutically effective amount is an amount of the subject pharmaceutical composition capable of preventing and/or curing (at least partially) a condition or disorder (e.g., an autoimmune diseases) and/or any complications thereof in a subject suffering from or having a risk of developing said condition or disorder. The specific amount/concentration of the active agent comprised may vary according to the method of administration and the need of a patient, and can be determined based on e.g., volume, viscosity, and/or body weight of a patient etc. For example, an appropriate dosage may be about 0.1 mg or 1 mg/kg/day to about 50 mg/kg/day; sometimes, the dosage can be even higher. It shall be understood that these specific doses may be conveniently adjusted by a skilled person in the art (e.g., a doctor or a pharmacist) based on conditions of a specific patient, formulation, and/or disease.

### Medical Use and Methods of Treatment

In another aspect, the present disclosure provides a use of the antibody or the antigen binding fragment thereof, the fusion protein, the protein conjugate, the isolated nucleic acid molecule or molecules, the vector or vectors, and/or the cell of the present disclosure in the manufacture of a medicament for preventing and/or treating pain comprising administering to a patient in need thereof a pharmaceutical composition of the present invention.

In a further aspect, the present application provides a method for preventing and/or treating a disease or disorder in a subject in need thereof, comprising administering to the subject an effective amount of the antibody or the antigen binding fragment the fusion protein, the protein conjugate, the isolated nucleic acid molecule or molecules, the vector or vectors, the cell, and/or the composition of the present disclosure, wherein the disease or disorder is a disease or disorder associated with an inappropriate expression or function of TrkA.

For example, the disease or disorder may comprise pain.

For example, the pain may comprise chronic pain.

For example, the disease or disorder may comprise chronic pain of nociceptive, non-nociceptive, traumatic, inflammatory, neuropathic, proliferative or mixed etiology. For example, the disease or disorder may comprise chronic pain or acute pain of musculoskeletal or neuropathic origin.

For example, the disease or disorder may comprise post-operative pain, rheumatoid arthritis pain, neuropathic pain and/or osteoarthritis pain.

In other embodiments, the pain is acute or chronic pain that is musculoskeletal or neuropathic in origin. Specific, non-limiting examples of pain that may be prevented and/or treated by the present application includes, for example, post-surgical pain, rheumatoid arthritis pain, neuropathic pain (includes radicular pain CLBP, DNP, and LSR), and osteoarthritis pain (includes non-radicular pain). In some cases, the pain is chronic pain of both musculoskeletal and neuropathic in origin. In other embodiments, the pain is visceral pain (such as, for example, chronic prostatitis, interstitial cystitis or chronic pelvis pain).

For example, such disease or disorder may be pain associated with, but not limited to any of the following: pancreatitis, kidney stones, endometriosis, IBD, Crohn's disease, post-surgical adhesions, gall bladder stones, headaches, dysmenorrhea, musculoskeletal pain, sprains, visceral pain, ovarian cysts, prostatitis, cystitis, interstitial cystitis, post-operative pain, migraine, trigeminal neuralgia, pain from burns and/or wounds, pain associated with trauma, neuropathic pain, pain associated with musculoskeletal diseases, rheumatoid arthritis, osteoarthritis, ankylosing spondylitis, periarticular pathologies, oncological pain, pain from bone metastases, and/or HIV infection.

In another aspect, the present disclosure provides use of the antibody or the antigen binding fragment thereof, the fusion protein, or the protein conjugate in the manufacture of an agent for determining the presence and/or amount of TrkA and/or NGF/TrkA signaling pathway in a sample.

In another aspect, the present disclosure provides a method for determining the presence and/or amount of TrkA and/or NGF/TrkA signaling pathway in a sample, comprising: a) contacting the sample with the antibody or the antigen binding fragment, the fusion protein, or the protein conjugate of the present disclosure; and b) determining the presence and/or amount of the antibody or the antigen binding fragment, of the fusion protein, or of the protein conjugate bound to the sample.

### Examples

The following examples are set forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or see, second(s); min, minute(s); h or hr, hour(s); aa, amino acid(s); nt, nucleotide(s); i.m., intramuscular (ly); i.p., intraperitoneal (ly); s.c., subcutaneous (ly); and the like.

### Example 1 Generation of Anti-TrkA Monoclonal Antibody

### TrkA Recombinant Protein for Immunization

The extracellular domain (ECD) of human TrkA (hTrkA) and mouse TrkA (mTrkA) were synthesized, and subcloned in pcDNA3.1-based expression vector with C-terminus fused to a human fc tag (hFc). The resulted plasmids were transiently transfected into 293-F cells, and cultured for 6 days in a CO₂ incubator equipped with rotating shaker. The supernatant containing the recombinant protein was collected and cleared by centrifugation, then was purified with molecular sieve gel filtration chromatography.

### Immunization, Hybridoma Fusion and Cloning

A total of 16 BALB/c mice were immunized with the full length ECD proteins in three independent groups via iv, sc or im routes. The first (2 mice) and the second (6 mice) groups were immunized with hTrkA-ECD-hFc. The third group was immunized with hTrkA-ECD-hFc (4 mice) or mTrkA-ECD-hFc (4 mice). Antigen protein was mixed 1:1 with Freund's complete adjuvant (FCA) or Freund's incomplete adjuvant (FIA), and prepared a stable oil-in-water emulsion. FCA was only used with the first immunization. Subsequent immunizations were performed in PBS with FIA. Last immunization was done without using adjuvant. The dose for injection was 400-500 μg/mL/mouse. After immunization, samples of serum were taken to determine the antibody response (titer) to the immunizing antigen by indirect ELISA. Large volume of blood samples were collected when titer reached 1:32 or greater, and were placed at room temperature for 1 hr followed by storage at 4° C. overnight. Serum samples were then collected and stored at −20° C. When serum titer reached the level of 1:8100 or greater, the splenocytes were isolated and fused with SP2/0 myeloma cells by polyethylene glycol (group 1) or electro cell fusion (groups 2 and 3) methods to induce the cells to fuse, forming hybridomas. Fused cells were plated into 96-well plates, and grown in the presence of hypoxanthine-aminopterin-thymidine (HAT) for two weeks before polyclonal hybridomas were selected.

### Assess TrkA Binding Activity of Antibodies by ELISA

Hybridoma supernatants were initially screened with TrkA by indirect ELISA. Briefly, Dilute purified immunogens to a final concentration of 1 μg/mL in 0.1M NaHCO₃ buffer. Pipette 100 μL of diluted antigen to each well of a 96-well plate, and incubate at 4° C. overnight. Remove coating solution and add 100 μL blocking solution to each well and incubate for 1 hr at room temperature. Place the plate at 4° C. after removing blocking solution. 100 μL of diluted primary antibody (test sample) was pipetted in each well, and was incubated for 1 hr at room temperature. The content in the wells was removed and washed 3× with 300 μL PBST buffer. Flicked the plate and pat the plate to complete removal of PBS buffer. 100 μL of 1:2000 diluted Peroxidase-AffiniPure Goat Anti-Mouse IgG was pipetted in each well, and was incubated for 1 hr at room temperature. The content in the wells was removed and washed them 4× with 300 μL PBST. Pipette 100 μL of TMB substrate solution to the wells. After 15 min at room temperature, pipette 100 μL of stop solution (2M HCl) to the wells to terminate the reaction. Read the absorbance (OD: 450) of each well with a microplate reader and the data was analyzed.

### Subcloning

Hybridomas that showed positive binding in indirect ELISA were subsequently tested for functional assays to identify antibodies with good functional activity. The antibodies with positive functional activity were further subcloned by limiting dilution and characterized functional assays. The subclones selected through functional assays were defined as monoclonal antibodies and stored in 8% DMSO in culture medium with gradual freezing at −80° C. for 24 hrs and then in liquid nitrogen.

### Expression and Purification of Monoclonal Antibodies

To grow hybridoma cells for antibody production, the frozen positive hybridomas were quickly thawed in a 37° C. water bath and transferred to tubes with 10 ml pre-warmed (37° C.) serum-free DMEM culture medium. Centrifuge tubes at 2000 rpm for 5 min. Transfer pellet to cell culture bottle with complete DMEM medium and cells were cultured in a 5% CO₂ and 37° C. incubator for 4-7 days.

All cells were collected and was spined down at 2500-3000 rpm for 20 min at 4° C. Supernatant that containing antibody was collected and filtered with a double layer glass wool filter. Further purification of the antibody was performed by using affinity chromatography beads (Strep-Tactin®XTor rProtein A/G Bead) according to the manufacturer's instruction.

### TrkA Activity Measurement Using NFAT Reporter Assay

CellSensor® TrkA-NFAT-bla CHO-K1 cells (K1516, Life Technologies) contain a beta-lactamase reporter gene under control of the NFAT Response Element that has been stably integrated into CHO-K1 cells, along with human TrkA gene stably expressed. Therefore, once the cells were stimulated with nerve growth factor, TrkA signaling can be activated, which can be detected with a special fluorescent substrate of beta-lactamase. Cellsensor TrkA-NFAT-bla CHO-K1 cells in a density range of 312500 cells/ml were seeded in 384-well plates with 32 μl media per well 16 hrs before experiment. For agonist activity measurement, 8 μl of 5× stock solution of NGF was added to the positive control wells and 8 μl of 5× test antibody stock solution was added to the test wells. For antagonist activity measurement, 4 μl of 10× test antibody stock solution was added to the test wells. 4 μl of PBS was added to non-stimulated or cell free control wells. 4 μl of 10× K252A inhibitor solution was added to the antagonist positive control wells. Incubate the antagonist assay plate in a humidified 37° C./5% CO₂ incubator for 30 min and then add 4 μl of 10×NGF. Both agonist or antagonist plates were incubated in 37° C./5% CO₂ incubator for 5 hrs. 8 μl of 6× LiveBLAzer™-FRET B/G Substrate (CCF4-AM) mixture, prepared according to the manufacture's instruction was added to each well. The plate was covered to protect it from light and evaporation, and was incubated at room temperature for 2 hours. Cell-free wells were used as the background. FRET signals (405 nm excitation and 530 nm emission) were obtained from the microplate reader (C5, Biotek) for analysis of TrkA activities.

### Example 2 the Binding Activity of TrkA Antibody

The applicant provides a TrkA antibody termed 27A6 (the amino acid sequence of 27A6 heavy chain is as set forth as SEQ ID NO: 11; and the amino acid sequence of 27A6 light chain is as set forth as SEQ ID NO: 12, the amino acid sequence of 27A6 HCDR1-3 is as set forth in SEQ ID NO: 1-3 respectively, and the amino acid sequence of 27A6 LCDR1-3 is as set forth in SEQ ID NO: 4-6 respectively) obtained from Example 1, which lacks the engagement of P75 TR and competes with NGF binding.

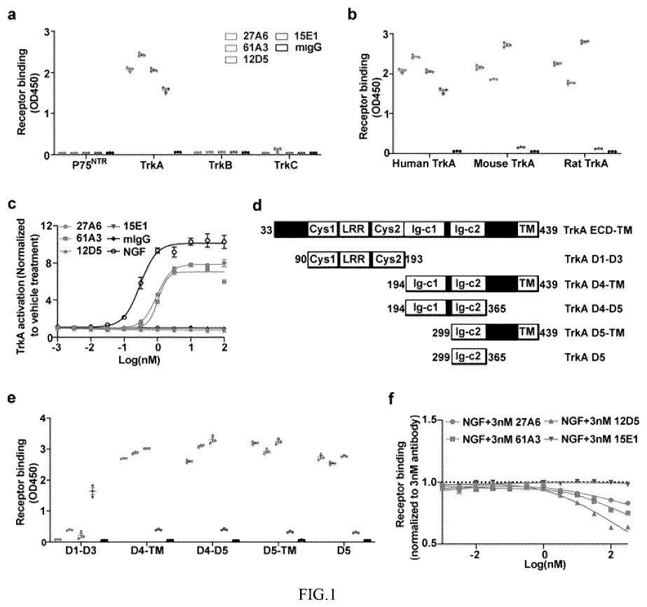

The cross-reactivities of TrkA antibodies to recombinant P75^{NTR}, TrkA, TrkB and TrkC proteins were tested by ELISA as described in Example 1 (n=3). Results are shown in FIG. 1a. The binding of TrkA antibodies to recombinant human, murine and rodent TrkA ECD proteins were tested by ELISA (n=3), and the results are shown in FIG. 1b. The dose response curve of TrkA activation by TrkA antibodies (n=3) is shown in FIG. 1c. The schematic representation of the domain composition of various TrkA constructs used in this study is shown in FIG. 1d. The binding abilities of TrkA antibodies to various epitopes on the TrkA ECD were measured by ELISA (n=3), and the results are shown in FIG. 1e. The abilities of TrkA antibodies to block NGF interaction with TrkA ECD were evaluated by competition ELISA (n=3), and the results are shown in FIG. 1f. Data are presented as mean±SEM.

Results show that 27A6 specifically binds to TrkA, and is capable of binding to human TrkA, mouse TrkA and rat TrkA, and 27A6 binds to D4-TM of human TrkA ECD.

### Example 3 the Effect of the TrkA Antibody on PC12 Cell Outgrowth

PC12 cells are pheochromocytoma cells, which express TrkA and P75^{NTR}. PC12 cells show sympathetic neuronal characteristics under the interaction of NGF and are the most common model for neurotoxicity, neuroprotection, neurosecretion, neuroinflammation and synaptogenesis researching.

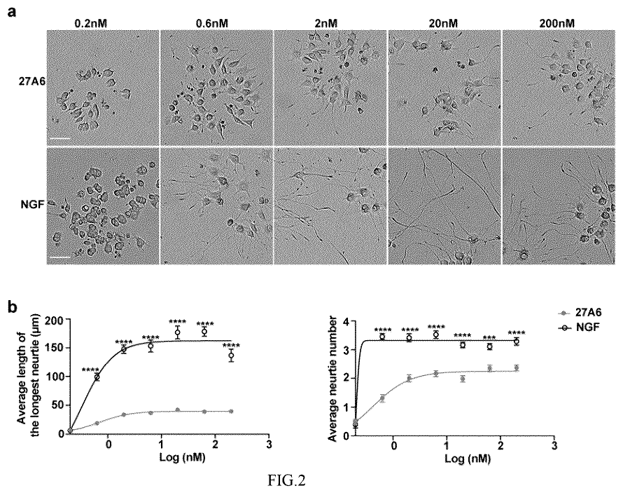

Parallel cultures were stimulated with either NGF or 27A6 at a range from 0.2 nM to 200 nM. The results are shown in FIGS. 2a-2b. In FIG. 2, 2a shows cell morphology images of PC12 cells at 9d were captured at a magnification of 20× via the high content screening system and representative images are shown. Scale bar: 50 μm; 2b shows average length of the longest neurite and average neurite number from (2a) were determined (n=55 cells). ****P<0.0001, two-way ANOVA was used. Data are presented as mean±SEM.

FIGS. 2a-2b shows that 27A6 maintains the inducing effect on neural protrusions in PC12 cells.

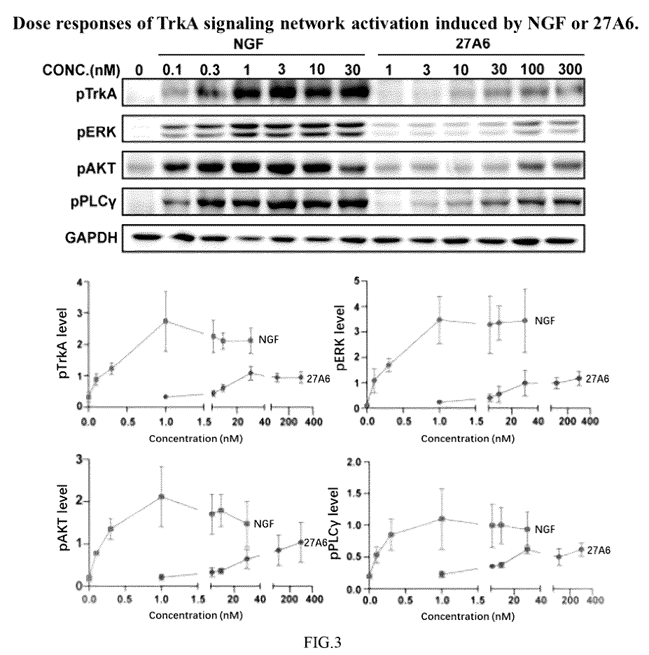

Dose responses of TrkA signaling network activation induced by NGF or 27A6 were tested. Data is shown in FIG. 3. FIG. 3 shows that 27A6 maintains the activation of TrkA signaling pathway in PC12 cells.

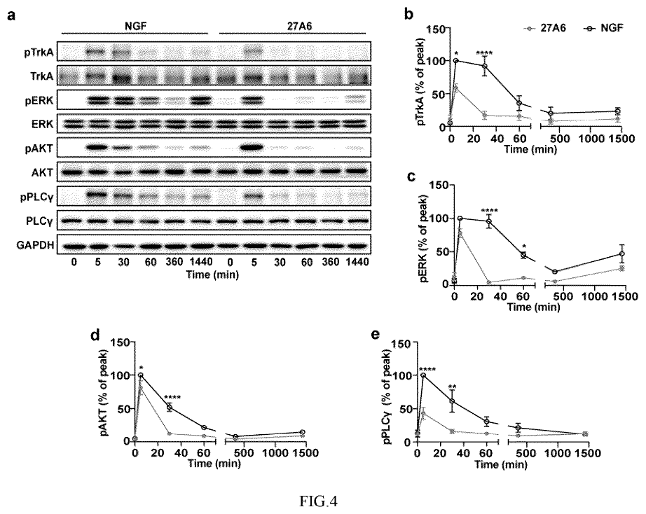

Serum-starved PC12 cells were either supplied with NGF (10 nM) or 27A6 (100 nM) at the indicated times or were left untreated. The results are shown in FIGS. 4a-4e. In FIG. 4, 4a shows total and phosphorylated protein levels were measured by immunoblotting of whole-cell lysates, with GAPDH as the indicator of cell number. Representative blots are shown; 4b shows quantitation of phosphorylated (Tyr490) TrkA levels; 4c shows quantitation of phosphorylated (Thy202/Tyr204) ERK levels; 4d shows quantitation of phosphorylated (Ser473) AKT levels; and 4e shows phosphorylated (Tyr783) PLCγ1 levels (all normalized to GAPDH and the peak value at 5 min of NGF) from (4a) (n=4 independent experiments). ****P<0.0001, **P<0.01, *P<0.05, two-way ANOVA was used. Data are presented as mean±SEM.

FIGS. 4a-4e show that 27A6 remains the transient activation of TrkA signaling pathway in PC12 cells.

### Example 4 the TrkA Antibody Reduces CFA-Induced Pain in Mice

To understand the pain relief effects of 27A6, complete Freund's adjuvant (CFA)-induced inflammatory pain mouse model was established and it was found intravenously injection of 27A6 significantly alleviated CFA-mediated thermal and mechanical thresholds reduction without boosting pain behavior in normal mice. Immunostaining data indicated that transient receptor potential vanilloid 1 (TRPV1) and calcitonin gene-related peptide (CGRP)-positive cells decrease in CFA injected mice after 27A6 treatment.

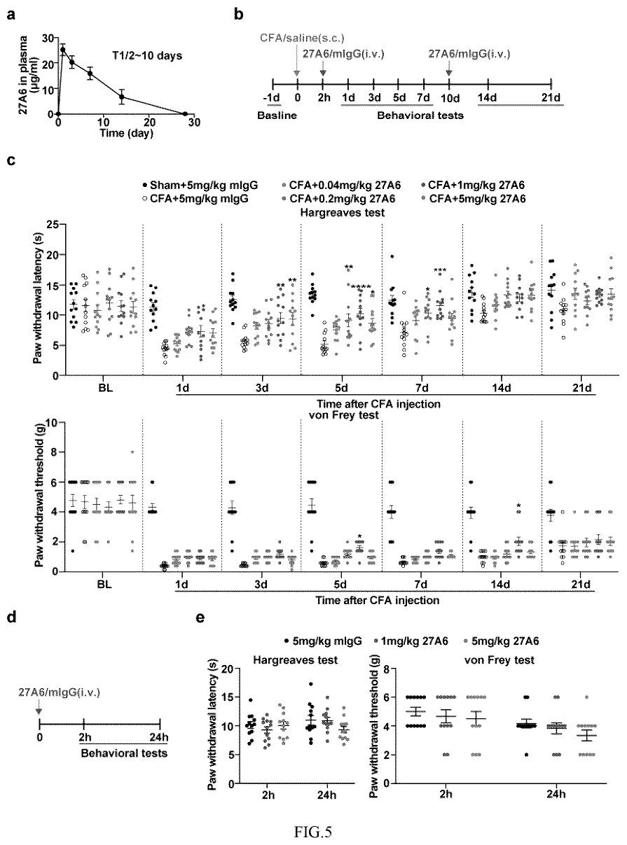

Results show that 27A6 mitigated CFA-induced pain sensitization but did not trigger pain in normal mice. The results are shown in FIGS. 5a-e. In FIG. 5, 5a shows the circulating half-life of 27A6 in plasma was determined by ELISA (n=4-6 mice); 5b shows the experimental design for studying analgesic effects of 27A6 in CFA-induced inflammatory pain; 5c shows the basis levels and therapeutic effects of 27A6 treatments (0.04-5 mg/kg) in thermal and mechanical pain thresholds accessed by Hargreaves test and von Frey test, respectively (n=12 mice). ****P<0.0001, ***P<0.001, **P<0.01, *P<0.05 versus CFA+5 mg/kg mouse IgG, two-way ANOVA was used; 5d shows the experimental design for studying painful effects of 27A6 in normal mice; and 5e shows the painful behavior in normal mice injected with 27A6 or mIgG (mouse IgG) tested by Hargreaves test and von Frey test (n=12 mice). Data are presented as mean±SEM.

In the therapeutic effect of 27A6 in thermal pain test, data shows that most groups of 27A6 shows great inhibition of pain, and most groups of 27A6 can maintain the therapeutic effect at Day 14.

In the therapeutic effect of 27A6 in thermal pain test, data shows that all groups of 27A6 shows great inhibition of pain, and most groups of 27A6 can maintain the therapeutic effect at Day 14.

### Example 5 the TrkA Antibody Promoted Membrane TrkA Internalization and Degradation

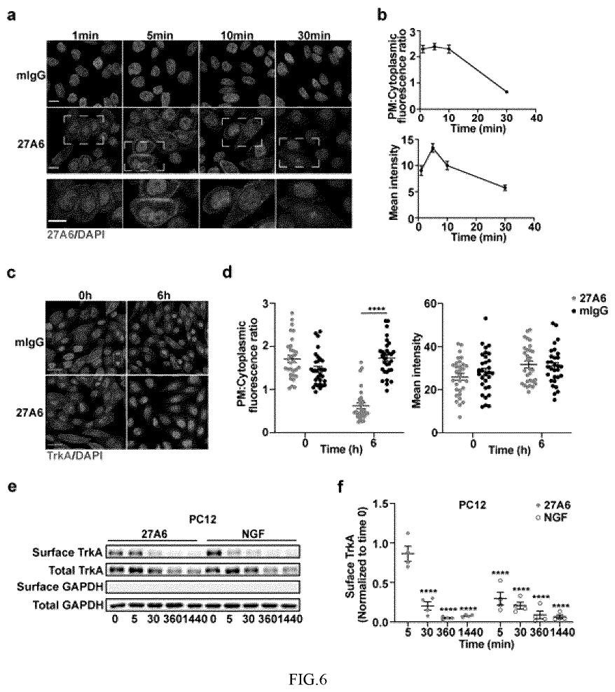

The applicant further tested if 27A6 can induce membrane TrkA internalization and degradation. The results are shown in FIGS. 6a-f. In FIG. 6, 6a shows CHO-TrkA cells were cultured on coverslips precoated with poly-D-lysine, and then were incubated with 27A6 (100 nM) or normal mIgG (100 nM) at serial time points from Imin to 30 min. Double staining for 27A6 and DAPI were captured at a magnification of 40× by the confocal laser scanning. The dashed white lines indicate the area detailed showed below. Representative images are shown. Scale bar: 10 μm; 6b shows the analyze result of plasma membrane (PM) to cytoplasm fluorescence ratios and the mean fluorescence intensity from 10A for 27A6 signal (n=30 cells); 6c shows cellular localization of TrkA after treatment with 27A6 (100 nM) or normal mIgG (100 nM) for 0 h or 6 h in CHO-TrkA cells. Double staining for TrkA and DAPI were captured at a magnification of 40× by the confocal laser scanning. Representative images are shown. Scale bar: 20 μm; 6d shows the analyze result of Plasma membrane (PM) to cytoplasm fluorescence ratios and the mean fluorescence intensity from 6c for TrkA signal (n=30 cells). ****P<0.0001, two-way ANOVA was used. 6E shows PC12 cells were left untreated or were treated with NGF (10 nM), or 27A6 (100 nM) for 5 min, 30 min, 6 h or 24 h. Total and surface TrkA levels were measured by immunoblotting of whole-cell lysates, with GAPDH as the indicator of cell numbers. Representative blots are shown. 6F shows the analyze result of surface TrkA levels by normalizing to GAPDH and the corresponding value at 0 min from 10E (n=4 independent experiments). ****P<0.0001, two-way ANOVA was used. Data are presented as mean±SEM.

Data shows that the TrkA antibody of the present disclosure can mediate self-internalization in TrkA-CHO cell, and promote the reduction of TrkA in TrkA-CHO cell membrane and PC12 cell membrane.

### Example 6 the TrkA Antibody Inhibits NGF-Dependent Signaling and Outgrowth

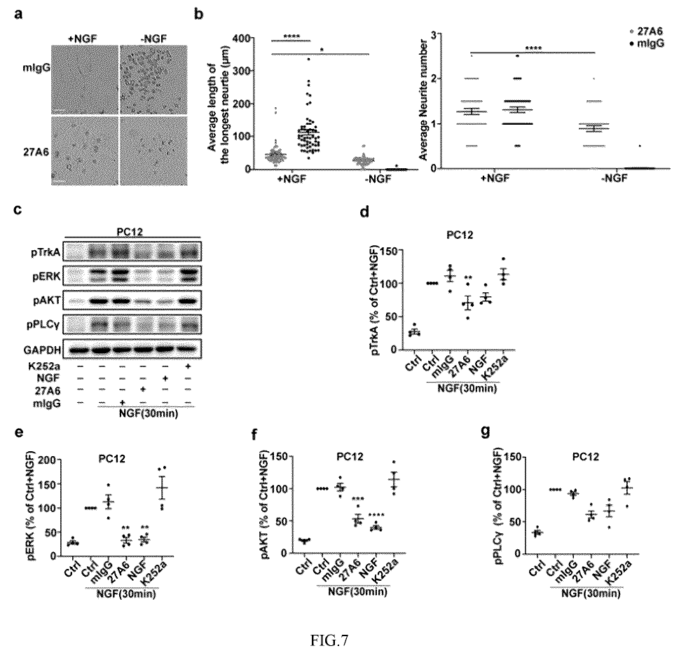

It was tested that if the TrkA antibody of the present disclosure can inhibit NGF-dependent signaling and outgrowth. The results are shown in FIGS. 7a-g. In FIG. 7, 7a shows PC12 cells were pretreated with either 27A6 (100 nM) or mIgG (100 nM) for 24 h, and then stimulated with NGF (1 nM). After 8 days, cell morphology images were captured, and representative images are shown. Scale bar: 50 μm; 7b shows average length of the longest neurite and average neurite numbers from 7a which were determined as indicated in the Methods section (n=35-55 cells); 7c shows PC12 cells were pretreated with PBS, NGF (10 nM), k252a (300 nM), 27A6 (100 nM) or mIgG (100 nM) for 24 h before stimulation with NGF (1 nM) or not for 30 min. Total and phosphorylated protein levels were measured with GAPDH as the indicator of cell number. Representative blots are shown; 7d shows quantitation of phosphorylated (Tyr490) TrkA levels; 7e shows quantitation of phosphorylated (Thy202/Tyr204) ERK levels; 7f shows quantitation of phosphorylated (Ser473) AKT levels and 7 g shows quantitation of phosphorylated (Tyr783) PLCγ1 levels (all normalized to GAPDH and the value of Ctrl+NGF from 7C (n=4 independent experiments). ****P<0.0001, ***P<0.001, **P<0.01, *P<0.05 versus mIgG+NGF, two-way ANOVA was used. Data are presented as mean±SEM.

Results show that the TrkA antibody of the present disclosure can inhibits NGF-dependent signaling and outgrowth.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. An antibody or an antigen binding fragment thereof, having one or more properties selected from the group consisting of:
1) is capable of specifically binding to TrkA;
2) is capable of competing with NGF for binding to TrkA;
3) is capable of activating TrkA signaling;
4) is capable of inducing membrane TrkA internalization;
5) is capable of mitigating CFA-induced pain sensitization; and
6) is capable of inhibiting NGF-dependent signaling.

2. The antibody or the antigen binding fragment of claim 1, wherein said antibody is selected from the group consisting of: a monoclonal antibody, a chimeric antibody, a humanized antibody, a fully human antibody and a bispecific antibody, wherein said antigen binding fragment is selected from the group consisting of: a Fab fragment, a Fab′ fragment, a F(ab)₂ fragment, a Fv fragment, a ScFv and a VHH.

3. (canceled)

4. (canceled)

5. The antibody or the antigen binding fragment of claim 1, which comprises a heavy chain CDR1 (HCDR1), a heavy chain CDR2 (HCDR2) and a heavy chain (HCDR3), said heavy chain CDR1 comprises an amino acid sequence as set forth in SEQ ID NO: 1, said heavy chain CDR2 comprises an amino acid sequence as set forth in SEQ ID NO: 2, and said heavy chain CDR3 comprises an amino acid sequence as set forth in SEQ ID NO: 3.

6. The antibody or the antigen binding fragment thereof of claim 1, wherein which comprises a light chain CDR1 (LCDR1), a light chain CDR2 (LCDR2) and a light chain CDR3 (LCDR3), said light chain CDR1 comprises an amino acid sequence as set forth in SEQ ID NO: 4, said light chain CDR2 comprises an amino acid sequence as set forth in SEQ ID NO: 5, and said light chain CDR3 comprises an amino acid sequence as set forth in SEQ ID NO: 6.

7. The antibody or the antigen binding fragment of claim 1, which comprises heavy chain CDRs1-3 and light chain CDRs1-3, said heavy chain CDR1 comprises an amino acid sequence as set forth in SEQ ID NO: 1, said heavy chain CDR2 comprises an amino acid sequence as set forth in SEQ ID NO: 2, said heavy chain CDR3 comprises an amino acid sequence as set forth in SEQ ID NO: 3, said light chain CDR1 comprises an amino acid sequence as set forth in SEQ ID NO: 4, said light chain CDR2 comprises an amino acid sequence as set forth in SEQ ID NO: 5, said light chain CDR3 comprises an amino acid sequence as set forth in SEQ ID NO: 6.

8. The antibody or the antigen binding fragment of claim 1, which comprises a heavy chain variable region (VH) and a light chain variable region (VL), said heavy chain variable region comprises an amino acid sequence as set forth in SEQ ID NO: 7, and said light chain variable region comprises an amino acid sequence as set forth in SEQ ID NO:8.

9. The antibody or the antigen binding fragment of claim 1, which comprises a heavy chain constant region.

10. The antibody or the antigen binding fragment of claim 9, wherein said heavy chain constant region comprises an amino acid sequence as set forth in SEQ ID NO: 9.

11. (canceled)

12. (canceled)

13. (canceled)

14. (canceled)

15. (canceled)

16. The antibody or the antigen binding fragment of claim 1, which comprises a light chain constant region.

17. The antibody or the antigen binding fragment of claim 16, wherein said light chain constant region comprises an amino acid sequence as set forth in SEQ ID NO: 10.

18. (canceled)

19. The antibody or the antigen binding fragment of claim 1, which comprises a heavy chain and a light chain, said heavy chain comprises an amino acid sequence as set forth in SEQ ID NO: 11, and said light chain comprises an amino acid sequence as set forth in SEQ ID NO: 12.

20. (canceled)

21. A fusion protein, comprising the antibody or the antigen binding fragment thereof of claim 1.

22. A protein conjugate, comprising the antibody or the antigen binding fragment thereof of claim 1.

23. Isolated nucleic acid molecule or molecules, encoding for the antibody or the antigen binding fragment thereof of claim 1.

24. Vector or vectors, comprising the isolated nucleic acid molecule or molecules of claim 23.

25. A cell, comprising the isolated nucleic acid molecule or molecules of claim 23.

26. A composition, comprising the antibody or the antigen binding fragment thereof of claim 1, and optionally a pharmaceutically acceptable excipient.

27. (canceled)

28. (canceled)

29. (canceled)

30. A method for preventing and/or treating a disease or disorder in a subject in need thereof, comprising administering to said subject an effective amount of the antibody or the antigen binding fragment thereof of claim 1, wherein said disease or disorder is a disease or disorder associated with an inappropriate expression or function of TrkA and/or TrkA/NGF signaling pathway.

31. The method of claim 30, wherein said disease or disorder comprises pain.

32. The method of claim 31, wherein said pain comprises chronic pain.

33. (canceled)
